(19) United States
(12) Patent Application Publication
(10) Pub. No.: US 2026/0036576 A1
(43) Pub. Date: Feb. 5, 2026

(54) Highly Sensitive Photothermal Microfluidic Thread-Based Multiplexed Immunosensor

(71) Applicant: Trustees of Tuffs College, Medford, MA (US)

(72) Inventors: Sameer Sonkusale, Lincoln, MA (US); Kawin Khachornsakkul, Malden, MA (US)

(21) Appl. No.: 19/286,388

(22) Filed: Jul. 31, 2025

Related U.S. Application Data
(60) Provisional application No. 63/678,280, filed on Aug. 1, 2024.

(51) Int. Cl. G01N 33/543; B01L 3/00; G01N 25/48; G01N 33/553

(52) U.S. Cl. CPC G01N 33/54346; B01L 3/5023; B01L 3/502715; G01N 25/48; G01N 33/54388; G01N 33/553; G01N 2400/28

(57) ABSTRACT

An assay device includes a thread that supports capillary flow of a sample from a sample zone to a test zone through a conjugation zone that has photothermal particles functionalized to bind to an analyte. The test zone is functionalized to trap those photothermal particles that have been bound to the analyte. Illuminating the test zone causes the photothermal particles to convert electromagnetic energy into thermal energy, thus a causing temperature rise, which a temperature sensor detects. This temperature rise results from photothermal conversion at the trapped photothermal particles.

### RELATED APPLICATIONS

This application claims the benefit of the Aug. 1, 2024 priority date of U.S. Provisional Application 63/678,280, the contents of which are incorporated herein by reference.

### BACKGROUND

The human immune system is a sophisticated interplay of diverse cellular components that cooperate to protect the human body against diseases. The cooperation requires communication among the various components.

Communication between the various parts of the immune system is typically carried out by special molecules that act as signals. Among these are small proteins called “cytokines.” These cytokines play an important role in regulating the operation of the immune system.

A difficulty that can arise in the course of an infection is an overproduction of cytokines. This overproduction, sometimes called a “cytokine storm,” can lead to widespread inflammation that can damage healthy tissues and organs.

Cytokines play a crucial role in human immunology by mediating communication between various cells to bolster the immune response. Monitoring cytokine levels within the body is of tremendous value for clinical diagnosis and prognosis. This arises in part because cytokines are associated with several diseases, including but not limited to inflammation, infection, injury, myocardial infarction, diabetes, Alzheimer's, Parkinson's disease, sepsis, asthma, heart disease, rheumatoid arthritis, Acquired Immune Deficiency Syndrome (AIDS), depression, and various cancers.

More recently, elevated serum cytokine levels have emerged as a crucial indicator for assessing the severity of COVID-19. Cytokine production has been found to sometimes significantly increases upon infection. This leads to a collapse of the immune system, often referred to as a “cytokine storm.” This phenomenon is not limited to COVID-19, It can occur in response to other infections and cancers, often with life-threatening consequences.

Cytokine biomarkers also play a vital role in a wide range of medical conditions, offering clinicians valuable insights for accurate diagnosis and informed treatment decisions.

The most common sensor for quantification of cytokines relies on immunological analysis, such as enzyme-linked immunosorbent assay and lateral flow immunoassay.

While such methods provide great selectivity and sensitivity, they can only be operated by a skilled-user requiring sufficient sample preparation and analysis time. Moreover, the instruments for quantitative monitoring using these methods might not be accessible to resource-limited settings for diagnosis and prognosis.

### SUMMARY

In one aspect, the invention contemplates the use of the photothermal effect for sensing one or more analytes. This is carried out by illuminating a sample carried by a thread with electromagnetic waves and measuring temperature change that results from the interaction of the sample with the electromagnetic waves. In preferred embodiments, the electromagnetic waves are coherent waves, particularly those in the visible range and those in the infrared range. A suitable source of such coherent electromagnetic waves is a laser.

Examples of analytes that are susceptible to sensing in this manner include cortisol, IL-6, CRP, TNF-alpha, IL-10, and IL-2.

In another aspect, the invention features a photothermal microfluidic-based thread analytical device for multiplexed sensing using threads that have been modified to include chitosan. Such a device is useful for quantitative monitoring of stress markers, cortisol and the inflammation markers, and interleukin-6. These markers are particularly useful for monitoring stress and inflammation as underlying causes for many chronic conditions, both mental and physical. The photothermal microfluidic-based thread analytical device can also be readily applied to monitoring other biomarkers.

In another aspect, a photothermal microfluidic-based thread analytical device includes a thread that has been pre-modified with chitosan and that has a sample zone, a capture zone, and a test zone. The resulting thread is assembled into a housing so that it can conveniently be illuminated by laser late to induce the photothermal effect and so that the resulting temperature change can easily be measured, for example through the use of a portable temperature sensor, such as a portable thermometer.

In operation, a sample is introduced into the sample zone via a sample injection port on the housing. The sample flows along both sides of the thread and encounters a photothermal agent in the test zone. A particularly useful photothermal agent takes the form of photothermal nanoparticles, and in particular, gold nanoparticles. As used herein, “gold nanoparticles” refers to nanoparticles that include the element “gold” (i.e., “Au”) in metallic form and is not intended to mean nanoparticles that simply have the color commonly known as “gold.”

Preferably, the gold nanoparticles have been labelled with antibodies that correspond to the analytes to be detected. For example, to detect cortisol or interleukin, one labels the gold nanoparticles with anti-cortisol or anti-interleukin-6 antibodies. These stand ready to conjugate with any cortisol or interleukin that may be in the sample.

When illuminated by a laser operating at an energy that corresponds to the localized surface plasmon resonance of the photothermal agent, the photothermal agent locally heats the test zone. The local heating results in a local increase in temperature that can be measured by a temperature sensor, such as a portable temperature sensor.

The temperature rise provides an indirect indicator of a binding event at the test zone. As a result, the device monitors trace levels of such analytes (e.g., interleukin-6 and cortisol) in a human sample with remarkable accuracy and precision. In particular, sensitivity on the order of atto-grams per milliliter or atto-moles is achievable. This results in better performance than that achieved by colorimetric lateral flow immunoassay and even enzyme-linked immunosorbent assays.

In one aspect, the invention features an assay device that includes a thread, a laser, and a temperature sensor. The thread, which has a sample zone, a test zone, and a conjugation zone that is between the sample zone and the test zone, supports capillary flow of a sample from the sample zone to the test zone through the conjugation zone. The conjugation zone has photothermal particles that are functionalized to bind to the analyte and the test zone has been functionalized to trap those photothermal particles that have been bound to the analyte. The laser is disposed to illuminate the test zone so as to cause the photothermal particles to convert electromagnetic energy from the laser into thermal energy that causes a localized temperature rise at the test zone. This temperature rise is indicative of a quantity of photothermal particles trapped in the test zone. The temperature sensor is disposed to obtain a temperature of the test zone. In particular, the temperature sensor measures a local temperature increase that results from photothermal conversion at the trapped photothermal particles.

Some embodiments include a housing having a sample port over the sample zone, a laser port disposed to receive the laser to permit illumination of the test zone, and a temperature-sensor port disposed to receive the temperature sensor and to permit the temperature sensor to be in contact with the test zone.

Other embodiments also include a flexible plastic substrate. In such embodiments, the thread is integrated into the substrate. Among these embodiments are those in which the temperature sensor is disposed in an integrated circuit that is embedded in the substrate. The integrated circuit provides information indicative of the presence of the analyte in the sample to at least one of a display embedded in the substrate and to a wireless receiver outside of the substrate.

Embodiments further include those in which the thread is coated to preserve its hydrophilicity. Among these are embodiments in which the thread is coated with chitosan.

Embodiments include those in which the photothermal particles comprise gold nanoparticles, those in which the photothermal particles comprise particles that have been labelled to bind to the analyte, those in which the photothermal particles have been configured to bind with interleukin-6, those in which the photothermal particles comprise photothermal particles that have been configured to bind with cortisol, those in which photothermal particles have been configured to bind with interleukin-1 beta, those in which the photothermal particles have been configured to bind with CRP, and those in which the photothermal particles have been configured to bind with TNF-alpha.

Still other embodiments include those in which photothermal particles comprise gold nanoparticles that have been labelled with antibodies for the analyte and the test zone is configured to entrap gold nanoparticles that have antibodies that have encountered the analyte.

In other embodiments, the assay device carries out multiplexed analysis of plural analytes. In such embodiments, the thread is a first thread of a plurality of threads, each of which has its own conjugation zone and test zone. All threads share the sample zone. As a result, a sample dropped on the sample zone flows through all of the threads, Among these embodiments are those in which the temperature sensor is coupled to each of the test zones. Among these are embodiments in which a controller causes the laser to illuminate each of the test zones in sequence, in which case the temperature sensor obtains a corresponding sequence of measurements, each of which corresponds to one of a corresponding plurality of analytes.

Still other embodiments include those in which the thread has an absorbent pad at a distal end thereof to maintain capillary flow along the thread.

In another aspect, the invention features a method for assaying an analyte. Such a method includes placing a liquid on a sample zone of a thread. This thread includes a test zone and a conjugation zone that is between the sample zone and the test zone. The thread supports capillary flow of a sample from the sample zone to the test zone through the conjugation zone. Within the conjugation zone are photothermal particles that have been to bind to the analyte. Meanwhile, the test zone has been functionalized to trap those photothermal particles that have been bound to the analyte. The method continues with illuminating the test zone with electromagnetic radiation so as to cause the photothermal particles to convert electromagnetic energy into thermal energy that causes a localized temperature rise at the test zone. This temperature rise being indicative of a quantity of photothermal particles trapped in the test zone. The method then includes measuring a local temperature increase at the test zone. This temperature increase is that which results from photothermal conversion at the trapped photothermal particles.

These and other features of the invention will be apparent from the following detailed description and the accompanying figures, in which:

### BRIEF DESCRIPTION OF THE FIGURES

FIG. 1 shows an assay device for assay of plural analytes;

FIG. 2 shows a housing for an assay device for assay of two analytes;

FIGS. 3 and 4 show the base of the housing shown in FIG. 2 before and after assembly thereof;

FIGS. 5-7 shows an assay devices for a single analyte; and

FIG. 8 shows the thread in the assay device of FIG. 7.

### DETAILED DESCRIPTION

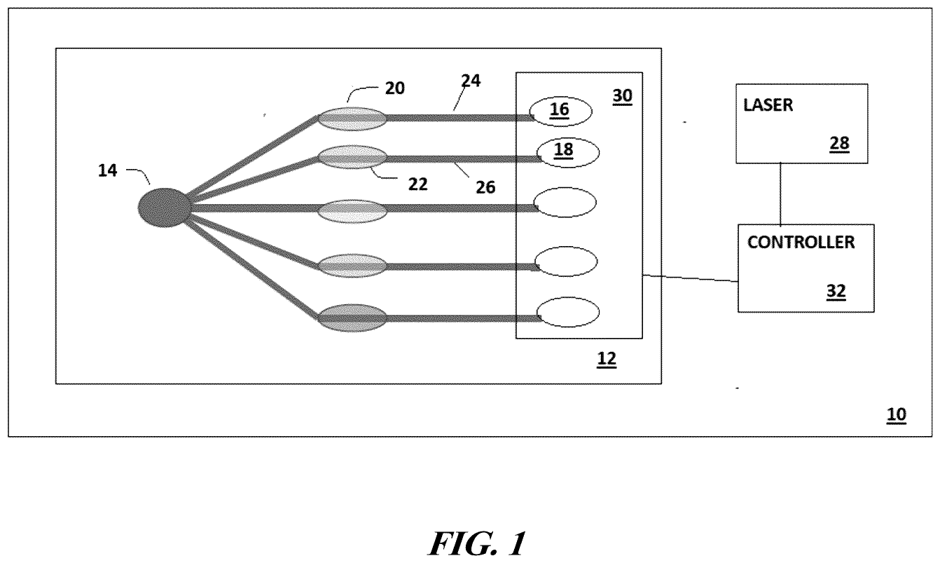

Referring to FIG. 1, assay device 10 for carrying out a lateral-flow immunoassay includes a substrate 12. A suitable substrate 12 is a flexible plastic strip.

The substrate 12 features a sample zone 14 connected to first and second test-zones 16, 18 via corresponding conjugate zones 20, 22 by corresponding threads 24, 26 that are embedded within the substrate. These threads 24, 26 support capillary flow between each test zone 16, 18 and the sample zone 14.

To support capillary flow, it is useful for the threads 24, 26 to be hydrophilic. One way to promote a thread's hydrophilicity is to expose its surface to a low-temperature plasma. This treatment removes non-cellulosic components from the thread's surface. In particular, it removes a superficial waxy barrier.

A difficulty that can arise with threads that have sustained such plasma treatment is that polar groups in the thread 24, 26 will slowly tend to reorient themselves in a way that reduces hydrophilicity. As a result, hydrophilicity decreases over time.

For some applications, the loss of hydrophilicity over time poses little difficulty. However, an assay device 10 may sit unused on a shelf for some time. As a result, by the time the assay device 10, the threads 24, 26 may no longer be sufficiently hydrophilic to support adequate capillary flow.

To promote the assay device's shelf life, it is particularly useful to further treat the threads 24, 26 to retain the hydrophilicity conferred upon them by plasma treatment. One method for doing so is to coat the thread 24, 26 with a suitable material, such as a polymer.

A particularly useful polymer coating is one obtained by deacetylation of chitin. The result of such a process is chitosan, the same material from which shells of many crustaceans are made. Chitosan is a practical choice in part because of the abundance of chitin in nature and because of chitosan's low toxicity, its biodegradability, its biocompatibility, and its stability. A suitable concentration of chitosan to use when treating a cotton thread is approximately twenty milligrams per milliliter.

Other polymers that are useful for coating the thread include carboxymethyl cellulose, polyvinyl alcohol, and polyvinyl chloride.

In those cases where the threads 24, 26 comprise cellulose fibers, it has been found that modifying cellulose fibers with chitosan further increases its wettability, thereby increasing flow rate through the thread 24, 26. The treatment also results in a more uniform surface. This, in turn, promotes accumulation of analytes and reagents in the thread's matrix. While the basis for this phenomenon is not completely understood, it is believed that it arises as a result of interaction between positively charged chitosan that has been adsorbed on the negatively charged cellulose on the thread 24, 26.

In some embodiments, threads 24, 26 are functionalized after coating the thread 24, 26 by immobilization of biorecognition elements on the surface thereof, for instance through covalent bonding with the chitosan. Such embodiments reduce the time required for analysis and also improve the ability to detect the relevant analytes.

An advantage of threads 24, 26 treated as described herein is their stability. Assay devices 10 that use such threads 24, 26 have been found to remain effective even after five months of storage. A chitosan coating thus provides two properties that cooperate synergistically in an assay device 10. First, the chitosan coating preserves wettability of the threads 24, 26 and second, the chitosan coating provides a suitable platform for immobilizing various biorecognition elements.

A sample dropped onto the sample zone 14 divides into first and second streams. The first stream flows through the first conjugate zone 20 until it reaches the first test-zone 16. Similarly, the second stream flows through the second conjugate zone 22 until it reaches the second test-zone 18. A suitable sample is a liquid taken from a patient. Examples of suitable liquids include blood serum, saliva, urine, and perspiration.

Each conjugate zone 20, 22 has, disposed thereon, an abundance of photothermal particles. Each such photothermal particle promotes the occurrence of the photothermal effect upon illumination by electromagnetic radiation of an appropriate energy by a laser 28. As used herein, “photothermal effect” refers to an energy-conversion process in which the energy carried by electromagnetic waves is converted into thermal energy as a result of interaction with the photothermal particles.

A suitable photothermal particle is one made from a material that displays a localized surface plasmon resonance. This results in significant conversion of energy carried by photons into energy carried by phonons, which in turn leads to efficient conversion of electromagnetic energy from light into thermal energy. Upon illumination by light of a resonant wavelength, electrons at the surface of the photothermal agent collectively oscillate and jump to an excited energy state. The energy that results from electron-electron scattering is then converted into thermal energy or heat. The resulting temperature increase is easily detected using a temperature sensor 30. Among the most effective photothermal agents having the foregoing property are gold nanoparticles.

The wavelength of the laser 28 plays an important role in the photothermal effect. The absorption of energy is highest at the resonance frequency of the photothermal particle due to the localized surface plasmon frequency. Consequently, the extent o which the desired photothermal effect manifests depends on the wavelength of incident radiation. In the case of gold nanoparticles, it has been discovered that a wavelength in the middle of the visible range, for example, at about 532 nanometers is suitable for monitoring both cortisol and interleukin-6. Other suitable wavelengths are at 532.2 nanometers and 528.9 nanometers.

Photothermal particles (e.g., gold nanoparticles) in different conjugate zones 20, 22 have been functionalized to bind to corresponding analytes. In those cases where an analyte is a protein, this can be carried out by conjugating recognition elements for that protein onto the photothermal particles that are in the conjugate zone 20, 22 for that protein. By functionalizing photothermal particles in different conjugate zones 20, 22 with recognition elements corresponding to different analytes, it becomes possible for the assay device 10 to carry out a multiplexed assay. Suitable recognition elements for binding to the analyte include antibodies, aptamers, nanobodies, peptides, lectins. In some embodiments, the binding element is a molecularly imprinted polymer that has been tuned to capture the relevant analyte.

Capillary flow through the first and second conjugate-zones 20, 22 transports the photothermal particles towards the first and second test-zones 16, 18. Analytes that are also present in the capillary flow will also interact with the corresponding functionalized photothermal particles to form immune complexes on the photothermal particles.

The first and second test zones 16, 18 have been functionalized to bind to the immune complex formed by the first and second analytes, respectively. As a result, photothermal particles that carry the immune complex are entrapped by the first and second test-zones 16, 18. Those that do not simply pass over the first and second test zones 16, 18.

The next step is to detect the presence of entrapped photothermal particles in the test zones 16, 18. This is carried out by illuminating each test zone 16, 18 with the laser 28. This induces the photothermal effect, which results in a localized increase in temperature that can be detected by the temperature sensor 30.

In some embodiments, the temperature sensor 30 comprises a thermocouple. In other embodiments, the temperature sensor 30 comprises an application specific integrated circuit that displays the relevant concentrations on a display incorporated into the substrate 12 or that includes a transmitter to transmit data to an external device, such as a smart phone. Embodiments of a suitable temperature sensor 30 further include an infrared sensor, such as an infrared camera, a sensor that senses based on one or more temperature dependent properties of a semiconductor device, such as a transistor or a diode, a temperature sensor, such as a bimetallic strip that senses based on one or more temperature dependent properties of matter, which in the case of this example would be a differential expansion coefficient,

A controller 32 causes the laser 28 to illuminate the first test-zone 16 and to then receive data from the temperature sensor 30. This temperature data is indicative of a rise in temperature at the first test-zone 16. The controller 32 uses this data to estimate the concentration of the first analyte.

The controller 32 then causes the laser 28 to illuminate the second test-zone 18 and to then receive data from the temperature sensor 30 indicative of a rise in temperature at the second test-zone 18. The controller 32 uses this data to estimate the concentration of the second analyte.

As a result of the foregoing operation, the same temperature sensor 30 can be used for all of the test zones 16, 18. This advantage arises because the laser 28 illuminates only one test zone 16, 18 at a time. Since the controller knows which test zone 16, 18 is being illuminated, it knows that the temperature measured by the temperature sensor 30 must be the result of illuminating that particular test zone 16, 18.

A microfluidic-based thread assay device 10 as described herein offers many advantages for carrying out immunoassays. These advantages include affordability, light weight, low consumption of reagent, and ease of modification.

The use of threads 24, 26, in particular, has an advantage over the more common use of paper. An initial advantage arises from mechanical strength. After all, a wet thread 24, 26 tends to have much greater mechanical strength than wet paper.

Yet another advantage of using threads 24, 26 is the ease with which threads 24, 26 can be designed and fabricated without the need for patterning a microchannel with a hydrophobic barrier. This advantage arises in part because, as a byproduct of the process by which threads are made, the fibers that form the thread also form self-contained microchannels for fluid transport via capillary force and for both chemical reactions and separations. Moreover, it is often the case that threads 24, 26 are easier to source and to assemble than paper-based strips for achieving different functionalities of a lateral flow immunoassay.

The assay device 10 has been described in terms of two analytes. However, the principles described herein are applicable to any number of analytes. The following examples show assay devices 10 that rely on the same principle of operation but with different form factors and for analysis of different numbers of analytes.

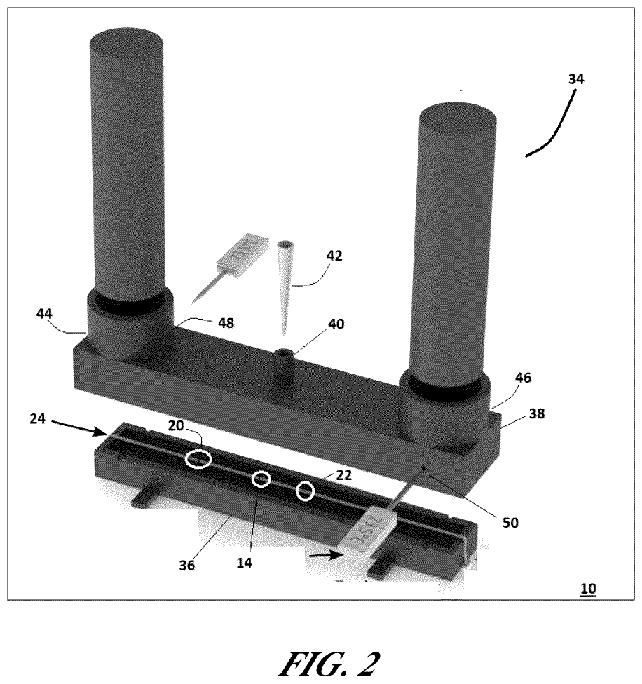

FIG. 2 shows a housing 34 to permit the convenient use of an assay device 10. The illustrated assay device 10 is one with a single thread for analysis of two analytes. However, a similar housing 34 can be used with minor modifications for other form factors. A housing 34 along the lines of the foregoing is manufactured by printing using a resin, preferably one that is black with a matte finish.

The housing 34 features a base 36 and a cover 38 that engages the base 36. A single thread 24 extends across a long axis of the base 36. The thread's sample zone 14 lies directly under a sample port 40 through which a pipette 42 drops the sample onto the sample zone 14.

Upon being introduced into the sample zone 14, the sample divides into two streams that flow in opposite directions away from the sample zone 14 past first and second conjugation zones 20, 22 and on to first and second test-zones 16, 18 that are disposed directly under corresponding laser ports 44, 46 in the cover 38. Temperature-sensor ports 48, 50 in the cover 38 permit entry of temperature probes 52, 54 to measure local heating caused by the photothermal effect at the test zones 16, 18.

In the foregoing embodiment, since there is one temperature probe 52, 54 for each test zone 16, 18, it is possible to carry out simultaneous measurements. The configuration shown in FIG. 2 is easily adaptable into a radial configuration in which multiple threads extend radially outward from the sample zone 14, thus permitting analysis of more than two analytes.

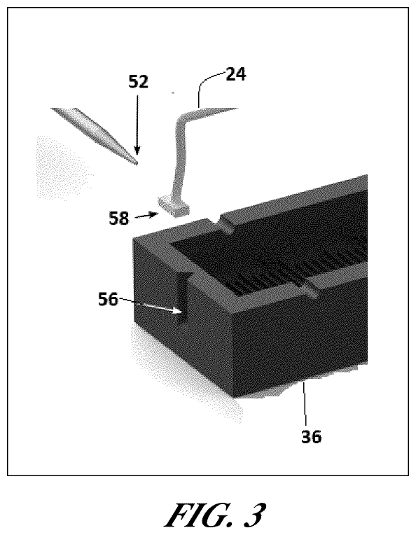

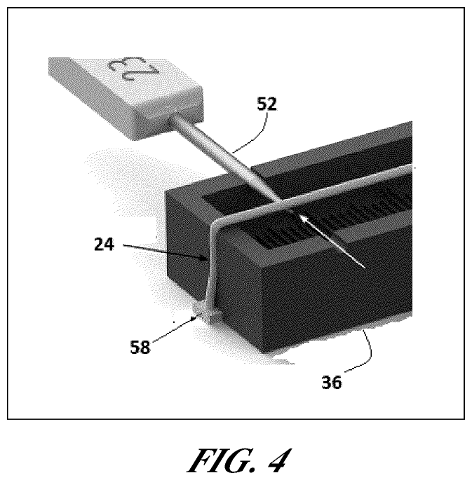

FIGS. 3 and 4 show closeups of the base 36 before and after assembly. As shown therein, the base 36 includes a groove 56 for engaging the thread 24. As shown in FIG. 3, the thread 24 includes an absorption pad 58 that absorbs liquid and thus maintains capillary flow for an extended period. The temperature probe 52 contacts the thread 24 at the test zone 16.

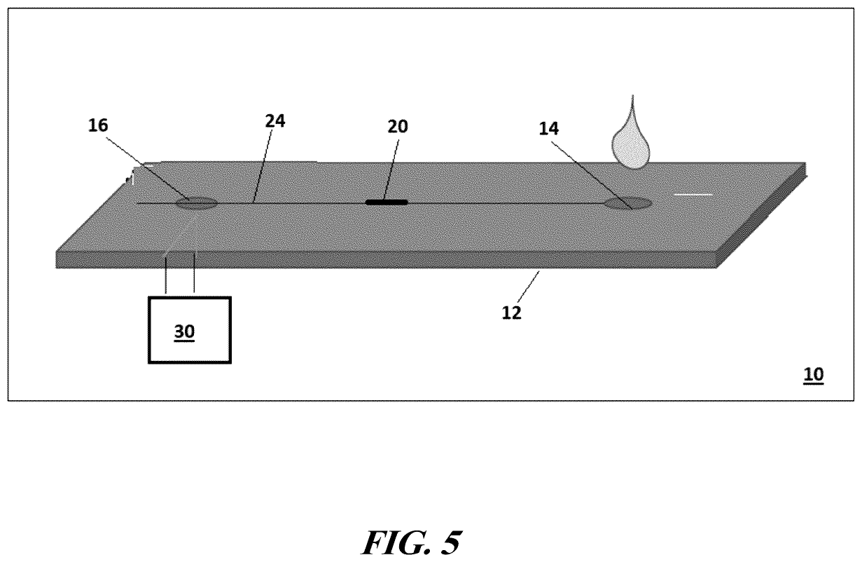

FIG. 5 shows an embodiment of an assay device 10 that is configured to detect only a single analyte. In the illustrated embodiment, the substrate 12 comprises a flexible plastic strip having a thread 24 embedded therein. The thread includes a sample zone 14, a single test zone 16, and a conjugate zone 20 therebetween. The substrate 12 also includes thermocouple wires 56 embedded therein for connection to a temperature sensor 30.

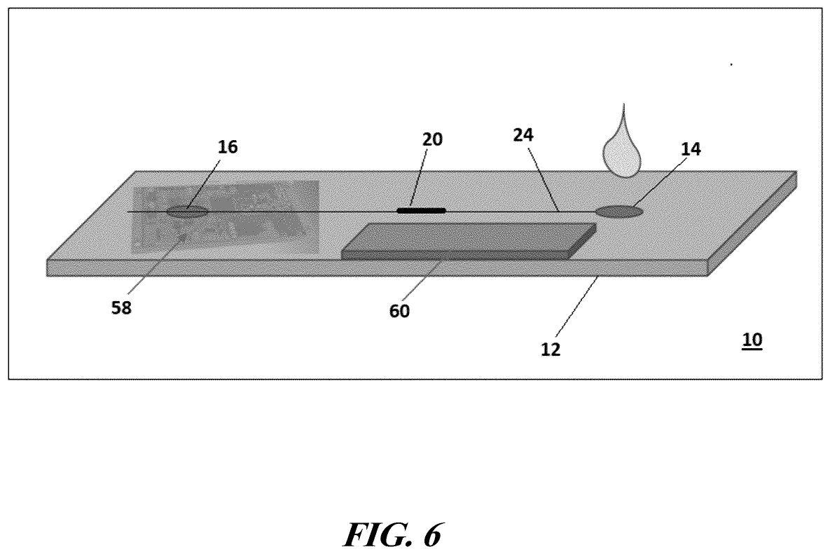

FIG. 6 shows a variation of the embodiment shown in FIG. 5 but with a temperature sensing integrated circuit 58 embedded within the substrate 12 and a display 60 connected to the integrated circuit 58 for displaying information indicative of a temperature rise caused by the photothermal effect. In an alternative embodiment, the integrated circuit 58 transmits measurement data wirelessly to a smartphone or other data processing device.

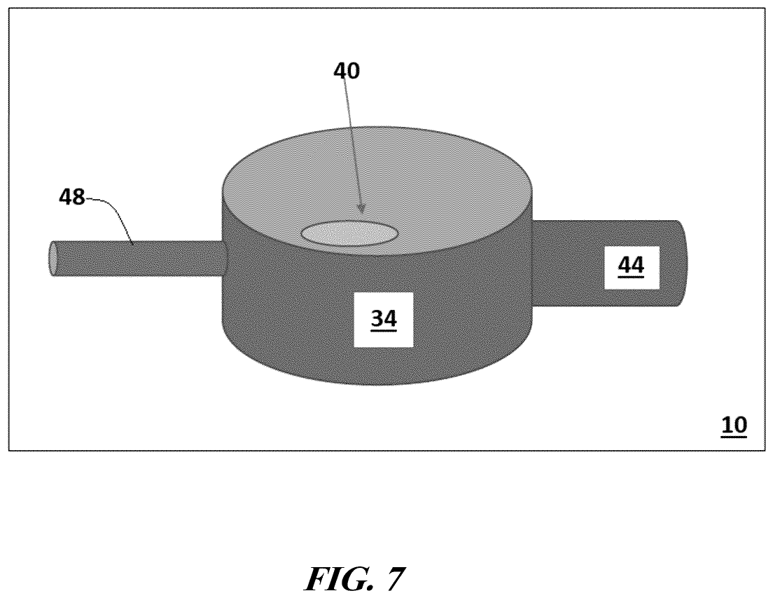

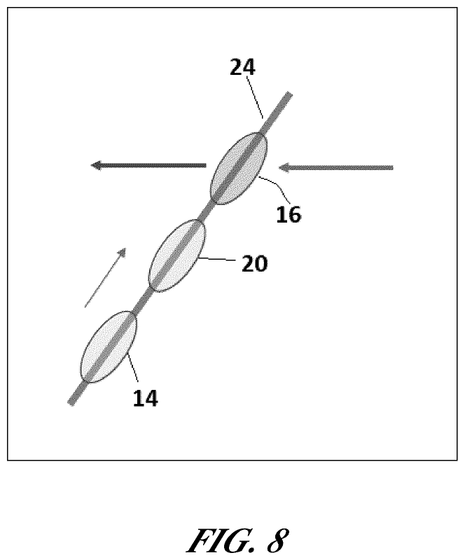

FIG. 7 shows a particularly compact handheld realization of the assay device 10 that comprises a housing 34 having a sample port 40, a laser port 44, and a temperature-sensor port 48. A thread 24, shown in FIG. 8, features a sample zone 14 that is disposed under the sample port 40 to receive the sample, a test zone 16 adjacent to both the laser port 44 and the temperature-sensor port 48 to receive laser radiation and to provide an output indicative of local heating, and a conjugate zone 20 therebetween, which holds the photothermal agent.

Having described the invention and a preferred embodiment thereof, what we claim as new and secured by Letters Patent is:

1. An apparatus for assaying an analyte, said apparatus comprising a thread, a laser, and a temperature sensor, wherein said thread comprises a sample zone, a test zone, and a conjugation zone that is between said sample zone and said test zone, wherein said thread supports capillary flow of a sample from said sample zone to said test zone through said conjugation zone, wherein said conjugation zone comprises photothermal particles, wherein said photothermal particles are functionalized to bind to said analyte, wherein said test zone has been functionalized to trap those photothermal particles that have been bound to said analyte, wherein said laser is disposed to illuminate said test zone so as to cause said photothermal particles to convert electromagnetic energy from said laser into thermal energy that causes a localized temperature rise at said test zone, said temperature rise being indicative of a quantity of photothermal particles trapped in said test zone, and wherein said temperature sensor is disposed to obtain a temperature of said test zone, and wherein said temperature sensor measures a local temperature increase that results from photothermal conversion at said trapped photothermal particles.

2. The apparatus of claim 1, further comprising a housing having a sample port over said sample zone, a laser port disposed to receive said laser to permit illumination of said test zone, and a temperature-sensor port disposed to receive a temperature sensor and to permit said temperature sensor to be in contact with said test zone.

3. The apparatus of claim 1, further comprising a flexible plastic substrate, wherein said thread is integrated into said substrate.

4. The apparatus of claim 1, further comprising a substrate and an integrated circuit embedded in said substrate, wherein said temperature sensor is disposed in said integrated circuit, and wherein said integrated circuit provides information indicative of the presence of said analyte in said sample to a display embedded in said substrate.

5. The apparatus of claim 1, further comprising a substrate and an integrated circuit embedded in said substrate, wherein said temperature sensor is disposed in said integrated circuit, and wherein said integrated circuit provides information indicative of the presence of said analyte in said sample to a wireless transmitter embedded in said substrate for transmission to a wireless receiver outside of said substrate.

6. The apparatus of claim 1, wherein said thread comprises a coating that preserves hydrophilicity thereof.

7. The apparatus of claim 1, wherein said photothermal particles comprise gold nanoparticles.

8. The apparatus of claim 1, wherein said photothermal particles comprise particles that have been labelled to bind to said analyte.

9. The apparatus of claim 1, wherein said wherein said photothermal particles have been configured to bind with interleukin-6.

10. The apparatus of claim 1, wherein said wherein said photothermal particles comprise photothermal particles that have been configured to bind with cortisol.

11. The apparatus of claim 1, wherein said photothermal particles have been configured to bind with interleukin-1 beta.

12. The apparatus of claim 1, wherein said photothermal particles have been configured to bind with CRP.

13. The apparatus of claim 1, wherein said photothermal particles have been configured to bind with TNF-alpha.

14. The apparatus of claim 1, wherein said photothermal particles comprise gold nanoparticles that have been labelled with antibodies for said analyte and said test zone is configured to entrap gold nanoparticles that have antibodies that have encountered said analyte.

15. The apparatus of claim 1, wherein said thread is a first thread of a plurality of threads, each of which comprises its own conjugation zone and test zone, wherein said threads all share said sample zone, whereby sample dropped on said sample zone flows through all of said threads, wherein said temperature sensor is coupled to each of said test zones.

16. The apparatus of claim 1, wherein said test zone is one of a plurality of test zones and said apparatus further comprises comprising a controller that causes said laser to illuminate each of said test zones in sequence, wherein said temperature sensor obtains a corresponding sequence of measurements, each of which corresponds to one of a corresponding plurality of analytes.

17. The apparatus of claim 1, wherein said thread comprises an absorbent pad at a distal end thereof to maintain capillary flow along said thread.

18. The apparatus of claim 1, further comprising a chitosan coating on said thread.

19. A method comprising assaying an analyte, said method comprising placing a liquid on a sample zone of a thread that comprises a test zone, and a conjugation zone that is between said sample zone and said test zone, wherein said thread supports capillary flow of a sample from said sample zone to said test zone through said conjugation zone, wherein said conjugation zone comprises photothermal particles, wherein said photothermal particles are functionalized to bind to said analyte, and wherein said test zone has been functionalized to trap those photothermal particles that have been bound to said analyte; illuminating said test zone with electromagnetic radiation so as to cause said photothermal particles to convert electromagnetic energy from said electromagnetic radiation into thermal energy that causes a localized temperature rise at said test zone, said temperature rise being indicative of a quantity of photothermal particles trapped in said test zone; and measuring a local temperature increase at said test zone, said temperature increase being a result of photothermal conversion at said trapped photothermal particles.
